(19) United States
(12) Patent Application Publication
(10) Pub. No.: US 2016/0244390 A1
(43) Pub. Date: Aug. 25, 2016

(54) Etherified Tri-Butylphenol Alkoxylates, Process For Their Preparation And Their Use In Crop Protection Agents

(71) Applicant: CLARIANT INTERNATIONAL LTD, Muttenz (CH)

(72) Inventors: Peter BAUR, Schondorf (DE); Robert MILBRADT, Wiesbaden (DE); Kai SCHROEDER, Burghausen (DE); Gerd SCHWEINITZER, Frankfurt am Main (DE)

(73) Assignee: Clariant International Ltd., Muttenz (CH)

(21) Appl. No.: 15/027,202

(22) PCT Filed: Oct. 6, 2014
(86) PCT No.: PCT/EP2014/002702

§ 371 Date: Apr. 4, 2016

(30) Foreign Application Priority Data
Oct. 7, 2013 (EP) 13187604.7

(51) Int. Cl. C07C 43/205; A01N 43/90; A01N 25/30; C07C 41/18

(52) U.S. Cl. CPC C07C 43/2055; C07C 41/18; A01N 43/90; A01N 25/30

(57) ABSTRACT

Etherified tri-butylphenol alkoxylates of the formula (I), wherein R represents an unbranched or branched, saturated or unsaturated C₁-C₄ alkyl, and A represents an ethylene group, a 1,2-propylene group, a 1,2-butylene group or mixtures of ethylene oxide groups and 1,2-propylene groups or mixtures of ethylene oxide groups and 1,2-butylene groups, and m is a number from 2 to 30, are suitable for improving the action of agrochemical active substances in and on plants.

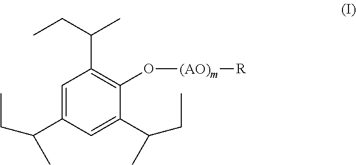

The invention relates to masked, i.e. etherified tributylphenol alkoxylates, to processes for preparing them, to their use for improving the effect of active agrochemical ingredients in and on plants, and to crop protection compositions which comprise the etherified tributylphenol alkoxylates.

The use of tributylphenol alkoxylates as additives for crop protection products is known. Not described to date, however, has been the employment of tributylphenol alkoxylates etherified on the hydroxyl group in crop protection products.

It has now been found that the effect of crop protection products is improved by particular etherified tributylphenol alkoxylates. Thus the etherified tributylphenol alkoxylates of the invention promote not only the retention of the crop protection agents on the plant, particularly on the leaves, but also the penetration of the active agrochemical ingredients contained in the crop protection products into the plant. This improvement in the properties is achieved even at low concentrations of etherified tributylphenol alkoxylates.

The invention accordingly provides etherified 2,4,6-tri(2-butyl)phenol alkoxylates of the formula (I),

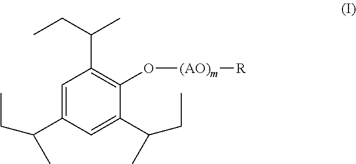

wherein
R is unbranched or branched, saturated or unsaturated C₁-C₄-alkyl, and
A is a 1,2-ethylene radical, a 1,2-propylene radical, a 1,2-butylene radical, or mixtures of ethylene and propylene radicals or mixtures of 1,2-ethylene and 1,2-butylene radicals, and
m is a number from 2 to 30.

Further provided by the invention is a process for preparing the etherified tributylphenol alkoxylates, comprising the step of
a) reacting a tributylphenol alkoxylate of the formula (II),

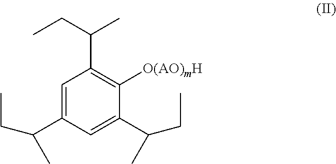

in which A and m have the definitions indicated above, with a C₁-C₄ alkyl halide, with catalysis by base.

Likewise provided by the invention is the use of the etherified tributylphenol alkoxylates of the formula (I) in crop protection compositions, more particularly for improving the effect of active agrochemical ingredients in and on plants.

Likewise provided by the invention is a crop protection composition comprising
A) one or more active agrochemical ingredients and
B) one or more etherified tributylphenol alkoxylates of the formula (I).

Further provided by the invention is the use of the etherified tributylphenol alkoxylates of the formula (I) as surfactant, as wetter and sticker, or as emulsifier.

A general definition of the etherified tributylphenol alkoxylates of the invention is provided by the formula (I). Preferred radical definitions in the formulae specified above and below are indicated hereinafter:

The symbols and indices in the formula (I) preferably have the following definitions:
R is preferably unbranched or branched, saturated C₁-C₄-alkyl.
A is preferably a 1,2-ethylene radical, a 1,2-propylene radical, or a mixture of 1,2-ethylene and 1,2-propylene radicals.
m is preferably a number from 2 to 20.

More preferably the symbols and indices in the formula (I) have the following definitions:
R is more preferably methyl or ethyl.
A is more preferably a 1,2-ethylene radical.
m is more preferably a number from 3 to 10.

Very preferably, R is methyl.

Very preferably, moreover, m is a number from 5 to 8.

The etherified tributylphenol alkoxylates of the invention can be prepared by known methods familiar to the skilled person. For this purpose, tributylphenol alkoxylates of the formula (II),

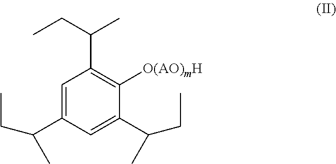

in which A and m have the definitions indicated above are reacted with a C₁-C₄-alkyl halide, preferably an alkyl chloride, with catalysis by base, preferably using NaOH.

The tributylphyl alkoxylates of the formula (II) used as a precursor are available commercially, under the Sapogenat brands from Clariant, for example.

They may be prepared from the corresponding phenol by reaction with alkylene oxides (EO, PO, BO or mixtures thereof) with ring-opening in the presence of catalysts. The process conditions, the process course and the catalyst are known to the skilled person.

Also provided by the invention is the use of the etherified tributylphenol alkoxylates of the formula (I) of the invention,

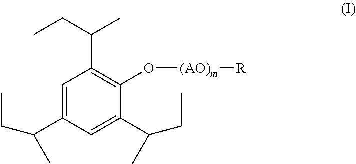

in which the symbols R, A and m have the definition indicated above for improving the effect of active agrochemical ingredients in and on plants.

Particularly preferred in this context is the improvement of the penetration of active agrochemical ingredients into plants and the improvement of the retention of active agrochemical ingredients on plants, especially on leaves. The method described in the literature (Baur et al., 1997, Pesticide Science 51, 131-152) can be used for determining these properties.

Also provided by the invention is the use of the etherified tributylphenol alkoxylates of the invention as surfactant, as wetter and sticker, and as emulsifier.

The compounds of the formula (I) are used individually or in the form of mixtures. Where reference is made in the description or the claims to etherified tributylphenol alkoxylates, the term relates expressly to individual compounds of the invention or to mixtures of two or more compounds of the invention.

In the context of the inventive use in crop protection compositions, the amount of one or more compounds of the formula (I) may vary within wide limits according to active ingredient and type of formulation. The compounds of the formula (I) can be used in all customary agrochemical formulations, preferably in those which are liquid. Also provided by the invention is the use of the etherified tributylphenol alkoxylates of the formula (I) for improving the effect at the level of the plant, as a tank-mix additive, meaning that the etherified tributylphenol alkoxylates are not added until directly before the application of a spray mixture produced from a concentrated formulation. In principle the compounds can also be incorporated into solid formulations.

The inventive use of the etherified tributylphenol alkoxylates of the formula (I) takes place, for example, in ready-to-apply crop protection compositions (spray mixtures), in which the amount of one or more etherified tributylphenol alkoxylates of the formula (I) is
0.01 to 3 wt %,
more preferably 0.01 to 1 wt %,
very preferably 0.02 to 0.5 wt %,
especially preferably 0.03 to 0.3 wt %.

Where a crop protection composition comprises two or more etherified tributylphenol alkoxylates, the quantity figure should be understood as the total amount of all etherified tributylphenol alkoxylates.

The radical definitions, value ranges and/or elucidations that are given above, whether those given generally or those given in ranges of preference, may also be combined as desired with one another, hence including combinations between the respective ranges and preference ranges.

Since the mechanism of action of the etherified tributylphenol alkoxylates as penetrants is fundamentally independent of the nature of the active agrochemical ingredient employed, their use is contemplated in all crop protection compositions comprising at least one active agrochemical ingredient whose biological activity may be enhanced by increased penetration into a crop plant or weed plant.

Since, furthermore, the mechanism of action of the etherified tributylphenol alkoxylates as retention promoters is fundamentally independent of the nature of the active agrochemical ingredient employed, their use is contemplated in all crop protection compositions comprising at least one active agrochemical ingredient whose biological activity may be enhanced by improved retention on a crop plant or weed plant.

“Active agrochemical ingredients” for the purposes of the invention include herbicides, fungicides, bactericides, insecticides, acaricides, molluscicides, nematicides, rodenticides, repellants, and phytohormones. Preference may be stated for fungicides, bactericides, insecticides, acaricides, nematicides, herbicides, plant growth regulators, plant nutrients, and repellants. The active agrochemical ingredients of the crop protection compositions of the invention are selected preferably from herbicides, insecticides, acaricides, and fungicides. An overview of the most important active agrochemical ingredients is found for example in “The Pesticide Manual” from the British Crop Protection Council, 16^{th} Edition 2012, Editor: C D S Tomlin.

Preferred fungicides are aliphatic nitrogen fungicides, amide fungicides such as acylamino acid fungicides or anilide fungicides or benzamide fungicides or strobilurin fungicides, aromatic fungicides, benzimidazole fungicides, benzothiazole fungicides, carbamate fungicides, conazole fungicides such as imidazoles or triazoles, dicarboximide fungicides, dithiocarbamate fungicides, imidazole fungicides, morpholine fungicides, oxazole fungicides, pyrazole fungicides, pyridine fungicides, pyrimidine fungicides, pyrrole fungicides, quinone fungicides.

Preferred herbicides are amide herbicides, anilide herbicides, aromatic acid herbicides such as benzoic acid herbicides or picolinic acid herbicides, benzoylcyclohexanedione herbicides, benzofuranyl alkylsulfonate herbicides, benzothiazole herbicides, carbamate herbicides, carbanilate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, imidazolinone herbicides, isoxazolidinone herbicides, nitrile herbicides, organophosphorus herbicides, oxadiazolone herbicides, oxazole herbicides, phenoxy herbicides such as phenoxy acetic acid herbicides or phenoxybutanoic acid herbicides or phenoxypropionic acid herbicides or aryloxyphenoxypropionic acid herbicides, pyrazole herbicides such as benzoylpyrazole herbicides or phenylpyrazole herbicides, pyridazinone herbicides, pyridine herbicides, thiocarbamate herbicides, triazine herbicides, triazinone herbicides, triazole herbicides, triazolone herbicides, triazolopyrimidine herbicides, uracil herbicides, urea herbicides such as phenylurea herbicides or sulfonylurea herbicides.

Preferred insecticides and acaricides are carbamate insecticides, such as benzofuranyl methylcarbamate insecticides or dimethylcarbamate insecticides or oxime carbamate insecticides or phenyl methylcarbamate insecticides, diamide insecticides, insect growth regulators, macrocyclic lactone insecticides such as avermectin insecticides or milbemycin insecticides or spinosyn insecticides, nereistoxin analog insecticides, nicotinoid insecticides such as nitroguanidine nicotinoid insecticides or pyridylmethylamine nicotinoid insecticides, organophosphorus insecticides such as organophosphate insecticides or organothiophosphate insecticides or phosphonate insecticides or phosphoramidothioate insecticides, oxadiazine insecticides, pyrazoline insecticides, pyrethroid insecticides such as pyrethroid ester insecticides or pyrethroid ether insecticides or pyrethroid oxime insecticides, tetramic acid insecticides, tetrahydrofurandione insecticides, thiazoline insecticides.

Examples of fungicides include:

(1) Ergosterol biosynthesis inhibitors, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifin, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxyphenoxy)-3,3-dimethylbutan-2-yl]1H-imidazole-1-carbothioate.
(2) Respiration inhibitors (respiratory chain inhibitors), for example bixafen, boscalid, carboxin, diflumetorim, fenfuram, fluopyram, flutolanil, fluxapyroxad, furametpyr, furmecyclox, isopyrazam mixture of the syn-epimeric racemate 1RS,4SR,9RS and of the anti-epimeric racemate 1RS,4SR,9SR, isopyrazam (anti-epimeric racemate), isopyrazam (anti-epimeric enantiomer 1R,4S,9S), isopyrazam (anti-epimeric enantiomer 1S,4R,9R), isopyrazam (syn-epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamid, 1-methyl-N-[2-1,1,2,2-tetrafluoroethoxy)phenyl]-3-(trifluoromethyl)-1H-pyrazole-4-carboxamide, 3-(difluoromethyl)-1-methyl-N-[2-1,1,2,2-tetrafluoroethoxy)phenyl]-1H-pyrazole-4-carboxamide, 3-(difluoromethyl)-N-[4-fluoro-2-1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazolin-4-amine, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.
(3) Respiration inhibitors (respiratory chain inhibitors) acting on complex III of the respiratory chain, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethhenyl]oxy}phenyl)ethylidine]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, methyl (2E)-2-{2-[({cyclopropyl[(4-methoxyphenyl)imino]methyl}sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide and (2R)-{2-(2-[(2,5-dimethylphenoxy)methyl]phenyl}-2-methoxy-N-methylacetamide.
(4) Mitosis and cell division inhibitors, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.
(5) Compounds with multisite activity, for example Bordeaux mixture, captafol, captan, chlorothalonil, copper preparations such as copper hydroxide, copper naphthenate, copper oxide, copper oxychloride, copper sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulfur and sulfur preparations, for example calcium polysulfide, thiram, tolylfluanid, zineb and ziram.
(6) Resistance inductors, for example acibenzolar-S-methyl, isotianil, probenazole and tiadinil.
(7) Amino acid and protein biosynthesis inhibitors, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.
(8) ATP production inhibitors, for example fentin acetate, fentin chloride, fentin hydroxide and silthiofam.
(9) Cell wall synthesis inhibitors, for example benthiavalicarb, dimethomorph, flumorph, iprovalicarb, mandipropamid, polyoxins, polyoxorim, validamycin A and valifenalate.
(10) Lipid and membrane synthesis inhibitors, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.
(11) Melanin biosynthesis inhibitors, for example carpropamid, diclocymet, fenoxanil, fthalide, pyroquilon, tricyclazole and 2,2,2-trifluoroethyl {3-methyl-1-[(4-methylbenzoyl)amino]butan-2-yl}carbamate.
(12) Nucleic acid synthesis inhibitors, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.
(13) Signal transduction inhibitors, for example chlozolinate, fenpiclonil, fludioxonil, iprodione, procymidone, quinoxyfen and vinclozolin.
(14) Decouplers, for example binapacryl, dinocap, ferimzone, fluazinam and meptyldinocap.
(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulfate, diphenylamine, ecomat, fenpyrazamine, flumetover, fluoromide, flusulfamide, flutianil, fosetyl-aluminum, fosetyl-calcium, fosetyl-sodium, hexachlorobenzene, irumamycin, methasulfocarb, methyl isothiocyanate, metrafenon, mildiomycin, natamycin, nickel dimethyldithiocarbamate, nitrothal-isopropyl, octhilinone, oxamocarb, oxyfenthiin, pentachlorophenol and salts thereof, phenothrin, phosphoric acid and salts thereof, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrin, tebufloquin, tecloftalam, tolnifanid, triazoxide, trichlamide, zarilamide, (3S,6S,7R,8R)-8-benzyl-3-[({3-[(isobutyryloxy)methoxy]-4-methoxypyridln-2-yl}carbonyl)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2,6-dimethyl-1H,5H-[1,4]dithiino[2,3-c:5,6-c′]dipyrrole-1,3,5,7(2H,6H)-tetrone, 2-[5-methyl-3-(triftuoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2 yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts thereof, 3-(4,4,5-trifluoro-3,3-dimethyl-3,4-dihydroisoquinolin-1-yl)quinoline, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidin-4-amine, 5-fluoro-2-[(4-methylbenzyl)oxy]pyrimidin-4-amine, 5-methyl-6-octyl[1,2,4]triazolo[1,5-a]pyrimidin-7-amine, ethyl (2Z)-3-amino-2-cyano-3-phenylprop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyrdin-2-yl)methyl]-2,4-dichloropyridine-3-carboxamide, N-[1-(5-bromo-3-chloropyridin-2-yl)ethyl]-2,4-dichloropyrndine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difiuoromethoxy)-2,3-difluorophenyl]methyl}-2-phenylacetamide, N-{(Z)-[(cyciopropylmethoxy)imino][6-(difluoromethoxy)-2,3-difluorophenyl]methyl}-2-phenylacetamide, N′-{4-[(3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, pentyl {6-[({[(1-methyl-1H-tetrazol-5-yl)(phenyl)methylidene]amino}oxy)methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl)(phenyl)methylene]amino}oxy)methyl]pyridin-2-yl}carbamate.
(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl)-2-yl)-3-(difluoromethyl)-1-methyl-1H-pyrazole-4-carboxamide, carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone, N-[2-(4-{[3-(4-chlorophenyl)prop-2-yn-1-yl]oxy}-3-methoxyphenyl)ethyl]-N2-(methylsulfonyl)valinamide, 4-oxo-4-[(2-phenylethyl)amino]butanoic acid and but-3-yn-1-yl {6-[({[(Z)-1-methyl-1H-tetrazol-5-yl)(phenyl)methylene]amino}oxy)methyl]pyridin-2-yl}carbamate.

Examples of bactericides include the following:

bronopol, dichlorophen, nitrapyrin, nickel dimethyldithiocarbamate, kasugamycin, octhilinon, furancarboxylic acid, oxytetracycline, probenazole, streptomycin, tecloftalam, copper sulfate and other copper preparations.

Examples of insecticides, acaricides and nematicides include the following:

(1) Acetylcholinesterase (AChE) inhibitors, such as carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or
organophosphates, for example acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel antagonists, such as, for example, cyclodiene organochlorines, for example chlordane and endosulfan; or phenylpyrazoles (fiproles), for example ethiprole and fipronil.
(3) Sodium channel modulators/voltage-dependent sodium channel blockers, such as, for example, pyrethroids, for example acrinathrin, allethrin, d-cis-trans-allethrin, d-trans-allethrin, bifenthrin, bioallethrin, bioallethrin-S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomers], deltamethrin, empenthrin [(EZ)-(1R)-isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans-isomer], prallethrin, pyrethrins (pyrethrum), resmethrin, silafluofen, tefluthrin, tetramethrin, tetramethrin [(1R)-isomers], tralomethrin, and transfluthrin; or DDT; or methoxychlor.
(4) Nicotinergic acetylcholine receptor (nAChR) agonists, such as, for example, neonicotinoids, for example acetamiprid, clothianidin, dinotefuran, imidacloprid, nitenpyram, thiacloprid, and thiamethoxam; or nicotine.
(5) Nicotinergic acetylcholine receptor (nAChR) allosteric activators, such as, for example, spinosyns, for example spinetoram and spinosad.
(6) Chloride channel activators, such as, for example, avermectins/milbemycins, for example abamectin, emamectin benzoate, lepimectin and milbemectin.
(7) Juvenile hormone imitators, such as, for example juvenile hormone analogs, for example hydroprene, kinoprene, and methoprene; or fenoxycarb; or pyriproxyfen.
(8) Active ingredients with unknown or non-specific mechanisms of action, such as, for example, alkyl halides, for example methyl bromide and other alkyl halides; or chloropicrin; or sulfuryl fluoride; or borax; or tartar emetic.
(9) Selective antifeedants, for example pymetrozine; or flonicamid.
(10) Mite growth inhibitors, for example clofentezine, hexythiazox and diflovidazin; or etoxazole.
(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.
(12) Oxidative phosphorylation inhibitors, ATP disruptors, such as, for example, dlafenthiuron; or
organotin compounds, for example azocyclotin, cyhexatin and fenbutatin oxide; or
propargite; or tetradifon.
(13) Oxidative phosphorylation decouplers acting by interrupting the H proton gradient, such as, for example, chlorfenapyr, DNOC and sulfluramid.
(14) Nicotinergic acetylcholine receptor antagonists, such as, for example, bensultap, cartap hydrochloride, thiocyclam, and thiosultap sodium.
(15) Chitin biosynthesis inhibitors, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.
(16) Chitin biosynthesis inhibitors, type 1, such as, for example, buprofezin.
(17) Molting disruptors, dipteran, such as, for example, cyromazine.
(18) Ecdysone receptor agonists, such as, for example, chromafenozide, halofenozide, methoxyfenozide and tebufenozide.
(19) Octopaminergic agonists, such as, for example, amitraz.
(20) Complex-III electron transport inhibitors, such as, for example, hydramethylnon; or acequinocyl; or fluacrypyrim.
(21) Complex-I electron transport inhibitors, for example
METI acaricides, for example fenazaquin, fenpyroximate, pyrimidifen, pyridaben, tebufenpyrad and tolfenpyrad; or rotenone (Denris).
(22) Voltage-dependent sodium channel blockers, for example indoxacarb; or metaflumizone.
(23) Inhibitors of acetyl-CoA carboxylase, such as, for example, tetronic and tetramic acid derivatives, for example spirodiclofen, spiromesifen and spirotetramat.
(24) Complex-IV electron transport inhibitors, such as, for example, phosphines, for example aluminum phosphide, calcium phosphide, phosphine and zinc phosphide; or cyanide.
(25) Complex-II electron transport inhibitors, such as, for example, cyenopyrafen.
(28) Ryanodine receptor effectors, such as, for example, diamides, for example chlorantraniliprole and flubendiamide.

Further active ingredients with unknown mechanism of action, such as, for example, amidoflumet, azadirachtin, benclothiaz, benzoximate, bifenazate, bromopropylate, chinomethionat, cryolite, cyantraniliprole (cyazypyr), cyflumetofen, dicofol, diflovidazin, fluensulfone, flufenerim, flufiprole, fluopyram, fufenozide, imidaclothiz, iprodione, pyridalyl, pyrifluquinazon and iodomethane; and also products based on Bacillus firmus (I-1582, BioNeem, Votivo):

Examples of herbicides include:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminocyclopyrachlor-potassium, aminocyclopyrachlor-methyl, aminopyralid, amitrole, ammonium sulfamate, ancymidol, anilofos, asulam, atrazine, aviglycine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, benzyladenine, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbaryl, carbetamide, carfentrazone, carfentrazone-ethyl, carvone, chlorocholine chloride, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlomitrofen, 4-chlorophenoxyacetic acid, chlorophthalim, chlorpropham, chlorthal-dimethyl, chiortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cloxyfonac, cumyluron, cyanamide, cyanazine, cyclanilide, cycloate, cyclosulfamuron, cycloxydim, cycluron, cyhalofop, cyhalofop-butyl, cyperquat, cyprazine, cyprazole, cytokinine, 2,4-D, 2,4-DB, daimuron/dymron, dalapon, daminozide, dazomet, n-decanol, desmedipham, desmetryn, detosyl-pyrazolate (DTP), diallate, diaminozide, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, diisopropylnaphthalene, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethyl naphthylacetate, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. O-(2,4-dimethyl-6-nitrophenyl) O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosuffuron-methyl-sodium, iofensulfuron, iofensulfuron-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazin-3-amine, MT-5950, i.e. N-[3-chloro-4-(1-methylethyl)phenyl]-2-methylpentanamide, NGGC-011, 1-naphthylacetic acid (NAA), naphthylacetamide (NAAm), 2-naphthoxyacetic acid, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitroguaiacolate, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasuffotole, pyrazolynate (pyrazolate), pyrazosulfuron, pyrazosulfuron-ethyl, pyrazoxyfen, pyribambenz, pyribambenz-isopropyl, pynribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasuffone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), suffentrazone, sulfometuron, sulfometuron-methyl, sulfosate (glyphosate-trimesium), sulfosulfuron, SW-065, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryne, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triafamone, triallate, triasulfuron, triaziflam, triazofenamide, tribenuron, tribenuron-methyl, tribufos, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vemolate, ZJ-0862, i.e. 3,4-dichloro-N-{2-[(4,6-dimethoxypyrimidin-2-yl)oxy]benzyl}aniline.

Examples of plant growth regulators further include natural plant hormones such as abscissic acid, jasmonic acid, salicylic acid and their esters, kinetin and brassinosteroids.

Examples of plant nutrients include conventional inorganic or organic fertilizers for providing plants with macronutrients and/or micronutrients.

Examples of repellents include diethyltolylamide, ethyihexanediol and butopyronoxyl.

“Crop protection compositions” for the purposes of the invention comprise the use form of the active agrochemical ingredients, examples being formulations and the spray mixture.

The invention further relates to crop protection compositions both in the form of the standard commercial formulations and of the use forms prepared from them, such as drench, drip and spray mixtures, comprising at least one etherified tributylphenol alkoxylate of the formula (I) and one or more active agrochemical ingredients.

The use forms optionally comprise further crop protection agents and/or effect-improving adjuvants, such as spreaders, for example alkylsiloxanes, and/or salts, for example organic or inorganic ammonium or phosphonium salts, for example ammonium sulfate or diammonium hydrogenphosphate, and/or humectants, for example glycerol, and/or fertilizers, for example ammonium-, potassium- or phosphorus-containing fertilizers.

Examples of conventional formulations are water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations optionally comprise, as well as one or more inventive active ingredients, further active agrochemical ingredients.

These are preferably formulations or use forms which comprise auxiliaries, for example extenders, solvents, spontaneity promoters, carriers, emulsifiers, dispersants, antifreezes, biocides, thickeners and/or further auxiliaries, for example further adjuvants. An adjuvant in this context is a component which enhances the biological effect of the formulation, without the component itself having any biological effect. Examples of adjuvants are the etherified tributylphenol alkoxylates of the invention as agents which promote retention, adhesion to the leaf surface, and penetration.

These formulations are produced in a known manner, for example by mixing the active ingredients with auxiliaries, for example extenders, solvents and/or solid carriers and/or further auxiliaries, for example surfactants. The formulations are produced either in suitable facilities or else before or during application.

Auxiliaries used may be further substances suitable for imparting special properties, such as certain physical, technical and/or biological properties, to the formulation of the active ingredient, or to the use forms prepared from these formulations (for example ready-to-use crop protection compositions such as spray mixtures or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and the ethers and esters thereof, ketones, for example acetone, methyl ethyl ketone, methyl isobutyl ketone or cyclohexanone, strongly polar solvents such as dimethyl sulfoxide, and also water.

In principle, it is also possible to use suitable solid carriers.

Liquefied gaseous extenders or solvents can also be used, in particular.

Examples of emulsifiers and/or foam formers, dispersants or wetting agents with ionic or nonionic properties, or mixtures of these surfactants, include salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkylsulfates, arylsulfonates, protein hydrolysates, lignosulfite waste liquors and methylcellulose. The presence of a surfactant is advantageous when one of the active ingredients and/or one of the inert carriers is insoluble in water and when application is effected in water.

Further auxiliaries which may be present in the formulations and the use forms derived therefrom include colorants such as inorganic pigments, for example iron oxide, titanium oxide and Prussian Blue, and organic colorants such as alizarin dyes, azo dyes and metal phthalocyanine dyes, and nutrients and trace nutrients such as salts of iron, manganese, boron, copper, cobalt, molybdenum and zinc.

Additional components may be stabilizers, such as low-temperature stabilizers, preservatives, antioxidants, light stabilizers, or other agents which improve chemical and/or physical stability. Foam formers or antifoams may also be present.

In addition, the formulations and the use forms derived therefrom may also comprise, as additional auxiliaries, stickers such as carboxymethyl cellulose, natural and synthetic polymers in the form of powders, granules or latices, such as gum arabic, polyvinyl alcohol and polyvinyl acetate, and also natural phospholipids such as cephalins and lecithins and synthetic phospholipids. Further auxiliaries may be mineral and vegetable oils.

Optionally, further auxiliaries may be present in the formulations and the use forms derived therefrom. Examples of such additives include fragrances, protective colloids, binders, adhesives, thickeners, thixotropic agents, stabilizers, sequestrants, complexing agents, humectants, spreaders. In general, the etherified tributylphenol alkoxylates of the formula (I) can be combined with any solid or liquid additive which is commonly used for formulation purposes.

Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulfate or diammonium hydrogenphosphate.

The formulations contain preferably between 0.00000001% and 98% by weight of active agrochemical ingredient or more preferably between 0.01% and 95% by weight of active agrochemical ingredient, more preferably between 0.5% and 90% by weight of active agrochemical ingredient, based on the weight of the formulation.

The active agrochemical ingredient content of the use forms prepared from the formulations can vary within wide limits. The active ingredient concentration of the use forms may typically be between 0.00000001% and 95% by weight of active agrochemical ingredient, preferably between 0.00001% and 1% by weight, based on the weight of the use form. Application is accomplished in a customary manner adapted to the use forms.

The content of the individual components in the inventive formulations can be varied within a relatively wide range.

The inventive formulations are produced, for example, by mixing the components, including the etherified tributylphenol alkoxylates of the formula (I), with one another in the particular ratios desired. If the active agrochemical ingredient is a solid substance, it is generally used either in finely ground form or in the form of a solution or suspension in an organic solvent or water. If the active agrochemical ingredient is liquid, there is frequently no need to use an organic solvent. It is also possible to use a solid active agrochemical ingredient in the form of a melt.

The temperatures can be varied within a particular range in the course of performance of the process. In general, working temperatures are between 0° C. and 80° C., preferably between 10° C. and 60° C.

In the performance of the process, the procedure is generally to mix the etherified tributylphenol alkoxylates of the formula (I) with one or more active ingredients and optionally with additives. The sequence in which the components are mixed with one another is arbitrary. Useful equipment for performance of the process is customary equipment which is used for production of agrochemical formulations.

Examples of administration forms include all the processes known as commonly used to the person skilled in the art: spraying, dipping, misting/fogging and a number of specific processes for direct treatment below or above ground of whole plants or parts (seed, root, stolons, stem, trunk, leaf), for example trunk injection in the case of trees or stem bandages in the case of perennial plants, and a number of specific indirect application processes.

The respective area- and/or object-based application rate of the crop protection compositions of a wide variety of different formulation types for control of the harmful organisms mentioned varies very greatly. In general, the application media known to the person skilled in the art to be commonly used for the respective field of use are used in the customary amounts for this purpose, for example several hundred liters of water per hectare in the case of standard spraying processes through a few liters of oil per hectare in the case of ‘ultra low volume’ aircraft application down to a few milliliters of a physiological solution in the case of injection processes. The concentrations of the inventive crop protection compositions in the particular application media therefore vary within a wide range and are dependent on the respective field of use. In general, concentrations known to the person skilled in the art to be commonly used for the respective field of use are used. Preferred concentrations are from 0.01% by weight to 99% by weight, more preferably from 0.1% by weight to 90% by weight.

According to the invention, it is possible to treat all plants and plant parts. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants. Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. Plant parts also include harvested material and vegetative and generative propagation material, for example cuttings, tubers, rhizomes, slips and seeds.

The preferred transgenic plants or plant cultivars (those obtained by genetic engineering) which are to be treated in accordance with the invention include all plants which, through the genetic modification, received genetic material which imparts particular advantageous useful traits to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, longer storage life and/or processability of the harvested products. Further and particularly emphasized examples of such properties are an improved defense of the plants against animal and microbial pests, such as against insects, mites, phytopathogenic fungi, bacteria and/or viruses, and also increased tolerance of the plants to certain herbicidally active ingredients. Examples of transgenic plants include the important crop plants, such as cereals (wheat, rice), maize, soybeans, potatoes, sugar beet, tomatoes, peas and other vegetable types, cotton, tobacco, oilseed rape, and also fruit plants (with the fruits of apples, pears, citrus fruits and grapes), particular emphasis being given to maize, soybeans, potatoes, cotton, tobacco and oilseed rape.

Preferred plants are those from the group of the useful plants, ornamental plants, turfgrass types, commonly used trees which are employed as ornamentals in public and domestic areas, and forestry trees. Forestry trees include trees for the production of timber, cellulose, paper and products made from parts of the trees.

The term useful plants as used here refers to crop plants which are employed as plants for obtaining foods, animal feeds, fuels or for industrial purposes.

The invention is illustrated in detail by the examples but is not restricted thereto.

### EXAMPLES

### Preparation of 2,4,6-tri-sec-butylphenol ethoxylate methyl ethers

### Example 1

### Preparation of 2,4,6-tributylphenoxy-ethoxy(6) methyl ether (TBPEM-6)

563 g of Sapogenat® T 060 (tributylphenol ethoxylate(6)) (Clariant) were conditioned to 60° C. under a nitrogen atmosphere and admixed with 60 g of solid NaOH. This was followed by evacuation and by metered introduction, over a period of three hours, of 75.7 g of MeCl, in the course of which there as a slight warming by about 5° C. After the addition had taken place, stirring was carried out at 80° C. for at least three hours, after which water of reaction and excess MeCl were removed by slow pressure reduction. Depending on the degree of conversion, the procedure described had to be repeated.

After the required conversion had been reached, water was added and the mixture was stirred at 80° C. for at least one hour, after which the batch was left to stand for phase separation. The organic phase was neutralized with phosphoric acid over a period of 30 minutes, dewatered, and filtered through Celite. The yield of TBPEM-6 was 577 g.

In a similar way, the methyl-etherified tributylphenol alkoxylate TBPEM-8 was obtained from Sapogenat T 080, ethoxylated with eight mols of ethylene oxide.

### Example 2

### Interfacial Activity

### Wetting Agent Effect

The dynamic surface tension was determined by the bubble pressure method (BP2100 tensiometer, Krúss) on a timescale relevant for spray application. The dynamic surface tension was measured for solutions of TBPEM-6 and TBPEM-8 in mains water at room temperature (22° C.) and at the typical spray mixture concentrations of 0.3, 1, and 3 g/l.

The low dynamic surface tensions, especially of TBPEM-6, show its excellent suitability as a rapid wetting agent, including its suitability as a sticking promoter, in the spray application of crop protection products. In the average time range of 100 milliseconds, the wetting effect was better (lower

| | Dynamic Conc | Surface tension [mN/m] | | |
|---|---|---|---|---|
| Substance | [g/L] | 20 ms | 100 ms | 200 ms |
| Sapogenat T 060 | 0.3 | 69.9 | 64.6 | 61.4 |
| Sapogenat T 060 | 1 | 67.3 | 58.6 | 53.2 |
| Sapogenat T 060 | 3 | 63.4 | 51.8 | 46 |
| TBPEM-6 | 0.3 | 70.3 | 65.0 | 62.8 |
| TBPEM-6 | 1 | 62.9 | 54.1 | 52.5 |
| TBPEM-6 | 3 | 55.5 | 42.9 | 41.8 |
| Sapogenat T 080 | 0.3 | 69.8 | 63.2 | 60.3 |
| Sapogenat T 080 | 1 | 64.7 | 57.3 | 52.2 |
| Sapogenat T 080 | 3 | 62.3 | 51.3 | 45.2 |
| TBPEM-8 | 0.3 | 69.1 | 61.7 | 58.6 |
| TBPEM-8 | 1 | 63.3 | 56.2 | 50 |
| TBPEM-8 | 3 | 59 | 49.7 | 44.6 |

### Example 3

### Percentage Penetration of Benzyladenine Through Apple Cuticles with and without TBPEM-6

The penetration of the growth promoter benzyladenine (benzylamonopurin) through leaf cuticles was measured at an active ingredient concentration of 0.2 g/l benzyladenine in acetone/mains water, with and without TBPEM-6, at different practically relevant concentrations, 5 hours and 24 hours after application.

In comparison to benzyladenine in solution in acetone/mains water, the penetration on addition of TBPEM-6 was more than seven times higher, measured after 5 and 24 hours. TBPEM-6 was added at a typical agivant concentration of 0.5, 1.0, and 3.0 g/l:

| Benzyladenine+ | 5 h | 24 h |
|---|---|---|
| Control (pure active ingredient) | 2.0 | 3.7 |
| 0.5 g/l TBPEM-6 | 18.0 | 26.1 |
| 1.0 g/l TBPEM-6 | 18.6 | 27.5 |
| 3.0 g/l TBPEM-6 | 45.5 | 58.9 |

### Example 4

The penetration of the herbicide atrazine through leaf cuticles was measured at an active ingredient concentration of 5 g/I atrazine (SC500), with and without the commercial additive Aureo® (rapeseed oil methyl ester) at 2.5 g/l at 1 g/l for the timepoints of 6, 18, and 48 hours after application.

In comparison to the atrazine SC formulation alone, the penetration measured at all three timepoints was at least four times to six times higher for addition at 1 g/l. The penetration of atrazine was, indeed, higher than that with the 2.5 times higher concentration of the commercial standard Aureo.

| Atrazine SC500 | g/l | 6 h | 18 h | 48 h |
|---|---|---|---|---|
| Control (pure active ingredient) | | 0.4 | 1.0 | 4.8 |
| +Aureo | 2.5 | 1.8 | 4.5 | 9.8 |
| +TBPEM-6 | 1 | 1.9 | 6.7 | 17.9 |

1. An etherified tributylphenol alkoxylate of the formula (I), 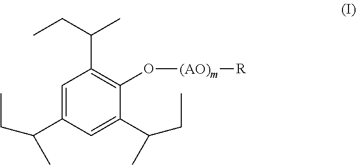
wherein
R is unbranched or branched, saturated or unsaturated C₁-C₄-alkyl, and
A is an ethylene radical, a 1,2-propylene radical, a 1,2-butylene radical, or mixtures of ethylene and 1,2-propylene radicals or mixtures of 1,2-ethylene and 1,2-butylene radicals, and
m is a number from 2 to 30.

2. An etherified tributylphenol alkoxylate as claimed in claim 1, in which
R is unbranched or branched, saturated C₁-C₄-alkyl,
A is a 1,2-ethylene radical, a 1,2-propylene radical, or mixtures of 1,2-ethylene and 1,2-propylene radicals, and
m is a number from 2 to 20.

3. An etherified tributylphenol alkoxylate as claimed in claim 1, in which
R is methyl or ethyl,
A is an ethylene radical, and
m is a number from 3 to 10.

4. An etherified tributylphenol alkoxylate as claimed in claim 1, in which R is methyl.

5. An etherified tributylphenol alkoxylate as claimed in claim 1, in which m is a number from 5 to 8.

6. A process for preparing an etherified tributylphenol alkoxylate comprising the step of
a) reacting a tributylphenol alkoxylate of the formula (II), 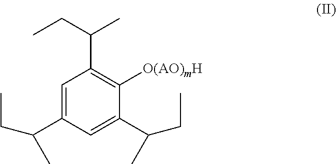
in which
A is an ethylene radical, a 1,2-propylene radical, a 1,2-butylene radical, or mixtures of ethylene and 1,2-propylene radicals or mixtures of 1,2-ethylene and 1,2-butylene radicals, and
m is a number from 2 to 30
with a C₁-C₄ alkyl halide, with catalysis by base.

7. A crop protection composition comprising an etherified tributylphenol alkoxylate of the formula (I) as claimed in claim 1.

8. A method for improving the effect of active agrochemical ingredients in and on plants comprising the step of adding an etherified tributylphenol alkoxylate of the formula (I) as claimed in claim 1 to the active agrochemical ingredients.

9. A method as claimed in claim 8, for improving the retention or the foliar uptake of active agrochemical ingredients.

10. A surfactant, wetter and sticker, or emulsifier comprising an etherified tributylphenol alkoxylate of the formula (I) as claimed in claim 1.

11. A crop protection composition comprising
A) at least one active agrochemical ingredients and
B) at least one etherified tributylphenol alkoxylate of the formula (I) as claimed in claim 1.
